(19) United States
(12) Patent Application Publication
(10) Pub. No.: US 2009/0274638 A1
(43) Pub. Date: Nov. 5, 2009

(54) COSMETIC USE OF A C-GLYCOSIDE DERIVATIVE IN COMBINATION WITH ASCORBIC ACID

(75) Inventors: Nathalie Pineau, Herblay (FR); Philippe Catroux, Guitres (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/306,566

(22) PCT Filed: Jul. 3, 2007
(86) PCT No.: PCT/FR2007/051587

§ 371 Date: Feb. 26, 2009

Related U.S. Application Data
(60) Provisional application No. 60/832,941, filed on Jul. 25, 2006.

(30) Foreign Application Priority Data
Jul. 3, 2006 (FR) 0606027

(51) Int. Cl. A61K 8/60; A61Q 19/00; A61Q 17/04

(52) U.S. Cl. 424/59; 514/23; 424/70.1

(57) ABSTRACT

The present invention relates to a cosmetic use of a synergistic combination of at least one C-glycoside derivative with at least ascorbic acid, or one of its derivatives or analogs, for preventively or curatively treating the signs of aging of body or facial skin, irrespective of whether they are chronobiological or photoinduced.

The present invention relates to the use, in the field of cosmetic and/or dermatological compositions, of a synergistic combination of at least ascorbic acid or a derivative or analog thereof with at least one C-glycoside derivative. The invention is also directed toward cosmetic and/or dermatological compositions comprising such a combination.

These combinations and compositions are preferentially intended for preventing and/or treating the cutaneous signs of aging and/or photoaging of the skin and in particular for increasing collagen synthesis.

Human skin consists of two tissues, one a surface tissue, the epidermis, and the other a deep tissue, the dermis.

Natural human epidermis is composed mainly of three types of cells, namely keratinocytes, which form the vast majority, melanocytes and Langerhans cells. Each of these three types of cells contributes, via its intrinsic functions, in the essential role played in the body by the skin, especially the role of protecting the body against external attacking factors (the climate, ultraviolet rays, tobacco, etc.), which is also known as the “barrier function”.

The dermis provides the epidermis with a solid support. It is also its nourishing element. It consists mainly of fibroblasts and of an extracellular matrix composed mainly of collagen, elastin and a substance known as ground substance. These components are synthesized by the fibroblasts. Leucocytes, mastocytes and tissue macrophages are also found therein. Finally, the dermis is interlaced with blood vessels and nerve fibers.

The cohesion between the epidermis and the dermis is provided by the dermo-epidermal junction. This is a complex region about 100 nm thick, which comprises the basal pole of the basal keratinocytes, the epidermal membrane and the sub-basal zone of the superficial dermis (Bernard P. Structure de la jonction dermo-épidermique. Objectif peau. [Structure of the dermo-epidermal junction. Objective: skin.] 2001, 68: 87-93). In structural terms, hemidesmosomes, in which are inserted keratin filaments (hemidesmosome-tonofilament complex), are distributed over the plasma membrane of the basal keratinocytes. As regards these hemidesmosome-tonofilament complexes, there are anchoring filaments present that pass through the epidermal basal membrane. The anchoring filaments are connected to the epidermal side of laminin 5. Finally, anchoring fibrils constitute the sub-basal network. These are curvilinear structures that arise and terminate on the deep face of the basal membrane and into which are inserted collagen I, III and V fibers. It has been shown that these anchoring fibrils, which may be clearly visualized by electron microscopy, are composed of type VII collagen. Type VII collagen is synthesized by the keratinocytes and the fibroblasts, but mainly by the keratinocytes (Aumailley M, Rousselle P. laminins of the dermo-epidermal junction. Matrix Biology, 1999, 18: 19-28; Nievers M, Schaapveld R, Sonnenberg A. Biology and function of hemidesmosomes. Matrix Biology, 1999, 18: 5-17).

Thus, collagens are major proteins of extracellular matrices. To date, 20 types of collagen have been identified and named from I to XX. Different families are distinguished among these types depending on the structures formed;
the family of fibrillar collagens (types I, II, III, V and XI) which form fibers;
the family of collagens forming the network of the basal membranes, which comprises type IV and type XVII collagen;
collagens forming hexagonal networks (type VIII and type X), pearled filaments (type VI), and FACITs (types IX, XII, XIV, XVI, XIX and XX);
the anchoring fibrils, which correspond to type VII;
multiplexins (types XV and XVII) and
collagen of type XIII, the precise functions of which are not known at the present time.

In the skin, the collagens mainly present throughout the dermis are the type I and III collagens which form the extracellular matrix of the entire dermis (these collagens constitute 70-80% of the dry weight of the dermis).

Moreover, collagens are not all synthesized by the same cell types; type I and III collagens are essentially produced by the dermal fibroblasts, whereas type VII collagen is produced by the epidermal keratinocytes. Finally, the regulation of their expression differs from one collagen to another, for example collagens I and VII are not regulated in the same way by certain cytokines; specifically, TNF-α and leukoregulin stimulate collagen VII and negatively regulate collagen I.

Finally, all collagen molecules are variants of a common precursor, which is the α chain of procollagen.

With age, collagen becomes thinner and wrinkles appear on the surface of the skin. Cutaneous aging is a genetically programmed mechanism. Moreover, certain environmental factors such as smoking and most particularly exposure to sunlight accelerate it. The skin thus has a much more aged appearance on the areas exposed to sunlight, such as the back of the hands or the face. Thus, these other factors also have a negative impact on the natural collagen of the skin.

Consequently, with regard to the crucial role of collagen, and more particularly of type VII collagen, on the cohesion between the epidermal and dermal tissues, and consequently on the integrity of the skin and its resistance to external attacking factors of mechanical type, stimulation of the synthesis of these various forms of collagen appears to be an effective means for overcoming the signs of cutaneous aging.

The aim of the present invention is, precisely, to propose a novel combination that may be used in cosmetics and pharmaceuticals for limiting aging of the skin, whether it is chronobiological or photoinduced aging, especially aging generated by a decrease in the skin's elasticity and/or by a degradation of the collagen in the structure of tissues.

The inventors have found, surprisingly, that a combination between ascorbic acid or a derivative or analog thereof and a C-glycoside derivative is capable of increasing the synthesis of procollagen 1 and thus of combating the signs of aging.

Sugars and sugar derivatives are products that have already been exploited for various purposes for the formulation of cosmetic compositions intended either for skincare or for caring for and/or washing keratin fibers. Thus, in WO 99/24009, D-xylose and derivatives thereof are proposed for the purposes of preparing cosmetic or pharmaceutical products aimed at improving the functionality of epidermal cells.

Among the sugars that may be used in the field, C-glycoside derivatives prove to be most particularly advantageous. Thus, certain C-glycoside derivatives have shown advantageous biological properties, in particular for combating aging of the epidermis and/or for combating skin dryness. Such compounds are described especially in document WO 02/051 828.

These compounds are more particularly represented by the formula:

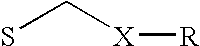

in which S represents a monosaccharide or a polysaccharide, R represents various linear or cyclic radicals and the group X may represent a group chosen from: —CO—, —CH(NR₁R₂)—, CHR′—, —C(═CHR′)— with R₁, R₂ and R′ possibly representing different radicals, including the hydroxyl radical.

Consequently, a first aspect of the invention relates to the cosmetic and/or dermatological use of a synergistic combination of at least ascorbic acid or a derivative or analog thereof with at least one C-glycoside derivative for preventively or curatively treating the signs of aging of bodily or facial skin, whether it is chronobiological or photoinduced aging, and especially aging generated by a decrease in the skin's elasticity and/or by a degradation of collagen in the structure of tissues.

Another subject of the invention is the use of a combination as defined above for preventively or curatively treating wrinkles and/or fine lines, wizened skin, lack of skin elasticity and/or tonicity, thinning of the dermis, degradation of collagen fibers, flaccid skin, thinned skin, and internal degradation of the skin following exposure to ultraviolet radiation.

Another subject of the invention is the use of a combination as defined above for inhibiting the activity of elastases and/or for limiting and/or combating the degradation of elastic fibers.

According to another of its aspects, the present invention relates to a cosmetic and/or dermatological composition comprising, in a physiologically acceptable medium containing an aqueous phase, at least one C-glycoside derivative in combination with at least ascorbic acid or a derivative or analog thereof.

According to yet another of its aspects, the present invention relates to an anhydrous cosmetic and/or dermatological composition comprising, in a physiologically acceptable medium, at least one C-glycoside derivative in combination with at least ascorbic acid or a derivative or analog thereof.

Another subject of the invention is a cosmetic process for treating bodily or facial skin, including the scalp, in which a cosmetic combination or composition as defined above is applied to the skin.

Another of the aspects of the invention relates to the use of a combination as defined above for the preparation of a dermatological composition for preventing and/or treating the signs of cutaneous aging and/or for stimulating the synthesis of collagen, in particular of procollagen 1.

According to yet another of its aspects, the invention relates to a therapeutic or nontherapeutic treatment process, in particular a cosmetic treatment process, for preventing and/or treating the signs of cutaneous aging, comprising the administration to an individual of a combination or a composition as defined above.

As emerges from the examples given hereinbelow, the inventors have found that the efficacy of a combination in accordance with the invention proves to be, against all expectation, markedly superior to that expected from the simple addition of the respective effects of each of the two compounds.

The term “cutaneous signs of aging” means any modification of the outer appearance of the skin caused by aging, whether it is chronobiological and/or photoinduced aging, for instance wrinkles and fine lines, wizened skin, lack of skin elasticity and/or tonicity, thinning of the dermis and/or degradation of collagen fibers, which leads to the appearance of flaccid and wrinkled skin; this expression also means any internal changes in the skin that are not systematically reflected by a changed outer appearance, for instance any internal degradation of the skin, particularly of elastin fibers, or elastic fibers, following exposure to ultraviolet radiation.

### C-GLYCOSIDE DERIVATIVES

A C-glycoside derivative that is suitable for use in the invention may be a compound of general formula (I) below:

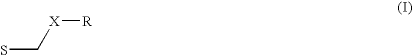

in which:
R represents:
   a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical;
   a saturated C₁-C₂₀ and in particular C₁-C₁₀ or unsaturated C₂-C₂₀ and in particular C₂-C₁₀ linear, or saturated or unsaturated, branched or cyclic C₃-C₂₀ and in particular C₃-C₁₀ hydrofluoroalkyl or perfluoroalkyl radical;
      the hydrocarbon-based chain constituting said radicals possibly being, where appropriate, interrupted with 1, 2, 3 or more heteroatoms chosen from:
an oxygen,
a sulfur,
a nitrogen, and
a silicon,
   and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
   with R₄ and R₅ possibly representing, independently of each other, a hydrogen atom or a saturated C₁-C₃₀ and in particular C₁-C₁₂ or unsaturated C₂-C₃₀ and in particular C₂-C₂₁ linear, or a saturated or unsaturated, branched or cyclic C₃-C₃₀ and in particular C₃-C₁₂ alkyl, perfluoroalkyl or hydrofluoroalkyl radical; or a C₆-C₁₀ aryl radical,
X represents a radical chosen from the groups:

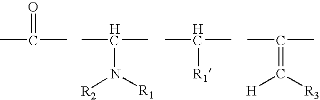

with R₁, R₂ and R₃ representing, independently of each other, a hydrogen atom or a radical R, with R as defined above, and R′₁ represents a hydrogen atom, an —OH group or a radical R as defined above, R₁ possibly also denoting a C₆-C₁₀ aryl radical;
S represents a monosaccharide or a polysaccharide comprising up to 20 sugar units and in particular up to 6 sugar units, in pyranose and/or furanose form and of L and/or D series, said mono- or polysaccharide possibly being substituted with a mandatorily free hydroxyl group, and optionally one or more optionally protected amine function(s), and
the bond S—CH₂—X represents a bond of C-anomeric nature, which may be α or β,
   and also the cosmetically acceptable salts thereof, the solvates thereof such as hydrates, and the isomers thereof.

In the context of the present invention, the term “halogen” means chlorine, fluorine, bromine or iodine.

The term “aryl” denotes an aromatic ring such as phenyl, optionally substituted with one or more C₁-C₄ alkyl radicals.

The term “C₃-C₈ cycloalkyl” denotes an aliphatic ring containing from 3 to 8 carbon atoms, for example including cyclopropyl, cyclopentyl and cyclohexyl.

Among the alkyl groups that are suitable for use in the invention, mention may be made especially of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, it is possible to use a C-glycoside derivative corresponding to formula (I) for which S may represent a monosaccharide or a polysaccharide containing up to 6 sugar units, in pyranose and/or furanose form and of L and/or D series, said monosaccharide or polysaccharide containing at least one hydroxyl function that is mandatorily free and/or optionally one or more amine functions that are mandatorily protected, X and R otherwise retaining all the definitions given above.

Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine and N-acetyl-D-galactosamine, and advantageously denotes D-glucose, D-xylose, N-acetyl-D-glucosamine or L-fucose, and in particular D-xylose.

More particularly, a polysaccharide of the invention containing up to 6 sugar units may be chosen from D-maltose, D-lactose, D-cellobiose, D-maltotriose, a disaccharide combining a uronic acid chosen from D-iduronic acid and D-glucuronic acid with a hexosamine chosen from D-galactosamine, D-glucosamine, N-acetyl-D-galactosamine and N-acetyl-D-glucosamine, an oligosaccharide containing at least one xylose advantageously chosen from xylobiose, methyl-β-xylobioside, xylotriose, xylotetraose, xylopentaose and xylohexaose and especially xylobiose, which is composed of two xylose molecules linked via a 1-4 bond.

More particularly, S may represent a monosaccharide chosen from D-glucose, D-xylose, L-fucose, D-galactose and D-maltose, especially D-xylose.

According to another embodiment of the invention, it is possible to use C-glycoside derivatives corresponding to formula (I) for which X represents a group chosen from —CO—, —CH(OH)—, —CH(NR₁R₂)— and —CH(R)—, in particular —CO—, —CH(OH)—, —CH(NH₂)—, —CH(NHCH₂CH₂CH₂OH)—, —CH(NHPh)- and —CH(CH₃)—, and more particularly a —CO—, —CH(OH)— or —CH(NH₂)— group, and preferentially a —CH(OH)— group, S and R otherwise conserving all of the definitions given above.

According to another embodiment of the invention, it is possible to use a C-glycoside derivative corresponding to formula (I) for which R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical; and optionally substituted as described above, S and X otherwise conserving all the definitions given above. Preferably, R denotes a linear C₁-C₄ and especially C₁-C₃ radical optionally substituted with —OH, —COOH or —COOR″₂, R″₂ being a saturated C₁-C₄ alkyl radical, especially ethyl, Preferentially, R denotes an unsubstituted linear C₁-C₄ and especially C₁-C₂ alkyl radical, in particular ethyl. Among the C-glycoside derivatives of formula (I) that are preferably used are those for which:
R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical, optionally substituted as described above;
S represents a monosaccharide as described above;
X represents —CO—, —CH(OH)—, —CH(NR₁R₂)— or —CH(R)—, as defined above.

Preferably, a C-glycoside derivative of formula (I) is used, for which:
R denotes a linear C₁-C₄ and especially C₁-C₃ radical, optionally substituted with —OH, —COOH or —COOR″₂, R″₂ being a saturated C₁-C₄ alkyl radical, especially ethyl;
S represents a monosaccharide as described above;
X represents a group chosen from —CO—, —CH(OH)—, —CH(NH₂)—, —CH(NHCH₂CH₂CH₂OH)—, —CH(NHPh)- and —CH(CH₃)—, and more particularly a —CO—, —CH(OH)— or —CH(NH₂)— group, and preferentially a —CH(OH)— group.

Preferentially, a C-glycoside derivative of formula (I) is used, for which:
R denotes an unsubstituted linear C₁-C₄ and especially C₁-C₂ alkyl radical, in particular ethyl;
S represents a monosaccharide as described above; especially D-glucose, D-xylose, N-acetyl-D-glucosamine or L-fucose, in particular D-xylose;
X represents a group chosen from —CO—, —CH(OH)— and —CH(NH₂)— and preferentially a CH(OH)— group.

The salts that are acceptable for the nontherapeutic use of the compounds described in the present invention comprise conventional nontoxic salts of said compounds such as those formed from organic or inorganic acids. Examples that may be mentioned include the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphonic groups. They may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

When the compound of formula (I) comprises an acid group, neutralization of the acid group(s) may be performed with a mineral base, such as LiOH, NaOH, KOH, Ca(OH)₂, NH₄OH, Mg(OH)₂ or Zn(OH)₂; or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made especially of amino-2-methyl-2-propanol, triethanolamine, dimethylamino-2-propanol or 2-amino-2-(hydroxymethyl)-1,3-propanediol. Mention may also be made of lysine or 3-(dimethyl-amino)propylamine.

The solvates that are acceptable for the compounds described in the present invention comprise conventional solvates such as those formed during the final step of preparation of said compounds due to the presence of solvents. Examples that may be mentioned include the solvates due to the presence of water or of linear or branched alcohols, for instance ethanol or isopropanol.

Among the C-glycoside derivatives of formula (I) used according to the invention, the ones that are most particularly considered are:
1. C-β-D-xylopyranoside-n-propan-2-one;
2. C-α-D-xylopyranoside-n-propan-2-one;
3. 1-[2-(3-hydroxypropylamino)propyl]-C-β-D-xylo-pyranose;
4. 1-[2-(3-hydroxypropylamino)propyl]-C-α-D-xylo-pyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-phenylaminopropane;
10. C-α-D-xylopyranoside-2-phenylaminopropane;
11. ethyl 3-methyl-4-(C-β-D-xylopyranoside)butyrate;
12. ethyl 3-methyl-4-(C-α-D-xylopyranoside)butyrate;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-phenylaminohexanoic acid;
20. 6-(C-α-D-xylopyranoside)-5-phenylaminohexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-phenylaminopentanoic acid;
30. 5-(C-α-D-xylopyranoside)-4-phenylaminopentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-phenylaminopropane;
46. 1-(C-α-D-fucopyranoside)-2-phenylaminopropane;
47. 1-(C-β-L-fucopyranoside)-2-phenylaminopropane;
48. 1-(C-α-L-fucopyranoside)-2-phenylaminopropane;
49. ethyl 3-methyl-4-(C-β-D-fucopyranoside)butyrate;
50. ethyl 3-methyl-4-(C-α-D-fucopyranoside)butyrate;
51. ethyl 3-methyl-4-(C-β-L-fucopyranoside)butyrate;
52. ethyl 3-methyl-4-(C-β-L-fucopyranoside)butyrate;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)-4-ketopentanoic acid;
72. 5-(C-α-L-fucopyranoside)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid;
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-fucopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-phenylaminopropane;
90. 1-(C-α-D-glucopyranosyl)-2-phenylaminopropane;
91. ethyl 3-methyl-4-(C-β-D-glucopyranosyl)butyrate;
92. ethyl 3-methyl-4-(C-α-D-glucopyranosyl)butyrate;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-phenylaminohexanoic acid;
100. 6-(C-α-D-glucopyranosyl)-5-phenylaminohexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-hydroxypentanoic acid;
109. 6-(C-β-D-glucopyranosyl)-5-phenylaminopentanoic acid;
110. 6-(C-α-D-glucopyranosyl)-5-phenylaminopentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-phenylaminopropane;
118. 1-(C-α-D-galactopyranosyl)-2-phenylaminopropane;
119. ethyl 3-methyl-4-(β-D-galactopyranosyl)butyrate;
120. ethyl 3-methyl-4-(α-D-galactopyranosyl)butyrate;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-phenylaminohexanoic acid;
128. 6-(C-α-D-galactopyranosyl)-5-phenylaminohexanoic acid;
129. 1-(C-β-D-galactopyranosyl)hexane-2,6-diol;
130. 1-(C-α-D-galactopyranosyl)hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-phenylaminopentanoic acid;
138. 6-(C-α-D-galactopyranosyl)-5-phenylaminopentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propane-2′-one;
146. 3′-(acetamido-C-α-D-glucopyranosyl)propane-2′-one;
147. 1-(acetamido-C-β-D-glucopyranosyl)-2-hydroxy-propane;
148. 1-(acetamido-C-β-D-glucopyranosyl)-2-aminopropane;
149. 1-(acetamido-C-β-D-glucopyranosyl)-2-phenylamino-propane;
150. 1-(acetamido-C-α-D-glucopyranosyl)-2-phenylamino propane;
151. ethyl 3-methyl-4-(acetamido-C-β-D-glucopyranosyl)-butyrate;
152. ethyl 3-methyl-4-(acetamido-C-α-D-glucopyranosyl)-butyrate;
153. 6-(acetamido-C-β-D-glucopyranosyl)-5-ketohexanoic acid;
154. 6-(acetamido-C-α-D-glucopyranosyl)-5-ketohexanoic acid,
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-phenylaminohexanoic acid;
160. 6-(acetamido-C-α-D-glucopyranosyl)-5-phenylaminohexanoic acid;
161. 1-(acetamido-C-β-D-glucopyranosyl) hexane-2,6-diol;
162. 1-(acetamido-C-α-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-phenylaminopentanoic acid;
170. 6-(acetamido-C-α-D-glucopyranosyl)-5-phenylaminopentanoic acid;
171. 1-(acetamido-C-β-D-glucopyranosyl)pentane-2,5-diol;
172. 1-(acetamido-C-α-D-glucopyranosyl)pentane-2,5-diol.

As nonlimiting illustrations of C-glycoside derivatives that are more particularly suitable for use in the invention, mention may be made especially of the following derivatives:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane,
1-(C-β-D-fucofuranosyl)propan-2-one,
1-(C-α-D-fucofuranosyl)propan-2-one,
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one,
C-β-D-maltopyranoside-2-hydroxypropane,
C-α-D-maltopyranoside-2-hydroxypropane, isomers thereof and mixtures thereof.

According to one embodiment, C-β-D-xylopyranoside-2-hydroxypropane or C-α-D-xylopyranoside-2-hydroxy-propane, and better still C-β-D-xylopyranoside-2-hydroxypropane, may advantageously be used for the preparation of a composition according to the invention.

According to one particular embodiment, the C-glycoside derivative may be C-β-D-xylopyranoside-2-hydroxypropane in pure form or in the form of a solution at 30% by weight of active material in a water/propylene glycol mixture (60%/40% by weight) such as the product sold by Chimex under the trade name Mexoryl SBB®.

Needless to say, according to the invention, a C-glycoside derivative corresponding to formula (I) may be used alone or as a mixture with other C-glycoside derivatives in any proportion.

A C-glycoside derivative that is suitable for use in the invention may especially be obtained via the synthetic method described in document WO 02/051 828.

The amount of C-glycoside derivative to be used in a composition according to the invention depends on the desired cosmetic or dermatological effect, and may thus vary within a wide range.

A person skilled in the art may readily determine the appropriate amounts, on the basis of his general knowledge.

A composition according to the invention may comprise a C-glycoside derivative in a proportion of about from 0.0001% to about 25% by weight of active material relative to the total weight of the composition, in particular from about 0.001% to about 10% by weight of C-glycoside relative to the total weight of the composition, and more particularly from about 0.05% to about 5% by weight of C-glycoside relative to the total weight of the composition.

Ascorbic Acid Derivatives or Analogs

According to the invention, ascorbic acid (or vitamin C) or an analog or derivative thereof is used.

Ascorbic acid is generally in L form, since it is usually extracted from natural products.

On account of its chemical structure (α-keto lactone) which makes it very sensitive to certain environmental parameters such as light, heat and aqueous media, it may be advantageous to use the ascorbic acid in the form of a derivative or an analog chosen, for example, from saccharide esters of ascorbic acid or metal salts of phosphoryl ascorbic acid, alkali metal salts, esters and sugars.

The saccharide esters of ascorbic acid that may be used in the invention are especially the glycosyl, mannosyl, fructosyl, fucosyl, galactosyl, N-acetylglucosamine and N-acetylmuramic derivatives of ascorbic acid, and mixtures thereof, and more especially ascorbyl-2 glucoside or 2-O-α-D-glucopyranosyl L-ascorbic acid or 6-O-β-D-galactopyranosyl L-ascorbic acid. The latter compounds and processes for preparing them are described in particular in documents EP-A-0 487 404, EP-A-0 425 066 and JP 05 213 736.

As regards the metal salt of phosphoryl ascorbic acid, it may be chosen from alkali metal, and especially sodium, ascorbyl phosphates, alkaline-earth metal ascorbyl phosphates and transition metal ascorbyl phosphates.

It is also possible to use ascorbic acid precursors such as active agent amides and active agent saccharide derivatives, which respectively involve proteases or peptidases and glycosidases as enzymes for releasing ascorbic acid in situ. Such compounds are described in patent EP 0 667 145.

The active agent saccharide derivatives are especially chosen from C₃ to C₆ saccharide derivatives. They are especially chosen from glucosyl, mannosyl, fructosyl, fucosyl, N-acetylglucosamine, galactosyl and N-acetylgalactosamine derivatives, N-acetylmuramic acid derivatives and sialic acid derivatives, and mixtures thereof.

The second ascorbic acid precursors may be chosen from derivatives that are hydrolyzed by other enzymes, for example by esterases, phosphatases, sulfatases, etc. According to the invention, the second active agent precursors may be chosen, for example, from phosphates; sulfates; palmitates; acetates; propionates; ferulates, and, in general, active agent alkyl or acyl esters; acyl or alkyl ethers. The acyl and alkyl radicals in particular contain from 1 to 30 carbon atoms.

In particular, the second precursor may be an ester derived from the reaction with a mineral acid such as a sulfate or a phosphate to react with a sulfatase or phosphatase on contact with the skin, and the second precursor may be an acyl or alkyl ester derived from the reaction with an organic acid, for instance palmitic acid, acetic acid, propionic acid, nicotinic acid, 1,2,3-propanetricarboxylic acid or ferulic acid to react with a specific skin esterase.

Other derivatives are described, for example, in patent EP 1 430 883.

The ascorbic acid analogs are, more particularly, its salts, especially alkali metal salts, for example sodium ascorbate, its esters, especially such as its acetic, propionic or palmitic esters, or its sugars, especially such as glycosyl ascorbic acid.

According to one preferred variant, it is ascorbic acid.

The effective amount of ascorbic acid, or of derivative or analog thereof, which may be used according to the invention is obviously that which is necessary to obtain the expected effect according to the invention and in particular the synergistic effect with regard to its combination with at least one C-glycoside derivative.

To give an order of magnitude, this amount preferentially represents from 0.001% to 20% of the total weight of the composition, preferentially from 0.1% to 15% of the total weight of the composition and advantageously from 3% to 10% of the total weight of the composition.

In addition, the composition of the invention is used for a time that is sufficient to obtain the expected effect according to the invention. To give an order of magnitude, this duration may be at least 15 days, but may also be more than 4 weeks, or even more than 8 weeks.

According to one particular embodiment, the ascorbic acid may be combined with the C-glycoside derivatives) in a mole ratio ranging from 1 to 10 mol of ascorbic acid or derivative or analog per 1 mol of C-glycoside derivative, in particular in a mole ratio ranging from 2 to 5 mol of ascorbic acid or derivative or analog per 1 mol of C-glycoside derivative.

According to one particular embodiment, the maximum concentration of C-glycoside derivative is used at a concentration of less than or equal to 3 mM, especially under the conditions as outlined in the example.

According to another embodiment, the weight ratio between the ascorbic acid and the C-glycoside derivative ranges between 0.6 and 200, for example between 0.6 and 50, between 50 and 100 or between 100 and 200, or even between 1 and 30, especially between 1 and 10.

The two types of compound as defined above may especially be used, in combination, in a composition that comprises a physiologically acceptable medium, especially in a cosmetic or pharmaceutical composition, in particular a dermatological composition, which therefore moreover comprises a cosmetically or pharmaceutically acceptable medium.

The physiologically acceptable medium in which the compounds according to the invention may be used, and also its constituents, their amount, the galenical form of the composition and the method for preparing it, may be chosen by a person skilled in the art on the basis of his general knowledge and as a function of the desired type of composition.

In general, this medium may be anhydrous or aqueous. It may thus comprise an aqueous phase and/or a fatty phase.

According to one embodiment, a subject of the invention is an aqueous composition comprising at least one C-glycoside derivative and at least ascorbic acid and/or a derivative or analog thereof as defined hereinabove.

The term “aqueous composition” means any composition comprising at least 5% by weight, preferably from 5% to 99% and even more preferentially from 20% to 99% by weight of water relative to the total weight of the composition.

Another subject of the invention is a cosmetic and/or dermatological anhydrous composition comprising, in a physiologically acceptable support, at least one C-glycoside or derivative and at least ascorbic acid and/or a derivative or analog thereof.

The term “anhydrous composition” means any composition comprising less than 5% water and more preferentially less than 1% water relative to the total weight of the composition.

For application to the skin, the composition may especially be in the form of an aqueous or oily solution; a dispersion of the lotion or serum type; emulsions of liquid or semiliquid consistency of the milk type obtained by dispersing a fatty phase in an aqueous phase (O/W) or conversely (W/O); suspensions or emulsions of soft consistency of the aqueous or anhydrous cream or gel type; microcapsules or microparticles; vesicular dispersions of ionic and/or nonionic type.

For application to the hair, the composition may be in the form of aqueous, alcoholic or aqueous-alcoholic solutions, in the form of creams, gels, emulsions or mousses; in the form of aerosol compositions also comprising a pressurized propellant.

When the composition is in aqueous form, especially in the form of an aqueous dispersion, emulsion or solution, it may comprise an aqueous phase, which may comprise water, a floral water and/or a spring water.

Said aqueous phase may also comprise one or more organic solvents such as a C₁-C₈ alcohol, especially ethanol, isopropanol, tert-butanol or n-butanol, or polyols such as glycerol, propylene glycol, butylene glycol, isoprene glycol, polyethylene glycol or polyol ethers.

When the composition according to the invention is in the form of an emulsion, it may also optionally comprise a surfactant, preferably in an amount of from 0.01% to 30% by weight, relative to the total weight of the composition. The composition according to the invention may also comprise at least one coemulsifier, which may be chosen from oxyethylenated sorbitan monostearate, fatty alcohols such as stearyl alcohol or cetyl alcohol, or fatty acid esters of polyols such as glyceryl stearate.

The composition according to the invention may also comprise a fatty phase, especially constituted of fatty substances that are liquid at 25° C., such as volatile or nonvolatile oils of animal, plant, mineral or synthetic origin; fatty substances that are solid at 25° C., such as waxes of animal, plant, mineral or synthetic origin; pasty fatty substances; gums; mixtures thereof.

The volatile oils are generally oils having, at 25° C., a saturating vapor pressure at least equal to 0.5 millibar (i.e. 50 Pa).

Among the constituents of the fatty phase, mention may be made of:
cyclic volatile silicones containing from 3 to 8 and preferably from 4 to 6 silicon atoms,
   cyclocopolymers of the dimethyl-siloxane/methylalkylsiloxane type,
linear volatile silicones containing from 2 to 9 silicon atoms,
volatile hydrocarbon-based oils, such as isoparaffins and especially isododecane and fluoro oils,
poly(C₁-C₂₀)alkylsiloxanes and especially those containing trimethylsilyl end groups, among which mention may be made of linear polydimethylsiloxanes and alkylmethylpolysiloxanes such as cetyl dimethicone (CTFA name),
silicones modified with optionally fluorinated aliphatic and/or aromatic groups, or with functional groups such as hydroxyl, thiol and/or amine groups,
phenyl silicone oils,
oils of animal, plant or mineral origin, and especially animal or plant oils formed from fatty acid esters of polyols, in particular liquid triglycerides, for example sunflower oil, corn oil, soybean oil, marrow oil, grapeseed oil, sesameseed oil, hazelnut oil, apricot oil, almond oil or avocado oil; fish oils, glyceryl tricaprocaprylate, or plant or animal oils of formula R₁COOR₂ in which R₁ represents a higher fatty acid residue containing from 7 to 19 carbon atoms and R₂ represents a branched hydrocarbon-based chain containing from 3 to 20 carbon atoms, for example purcellin oil; liquid paraffin, liquid petroleum jelly, perhydrosqualene, wheatgerm oil, beauty-leaf oil, sesameseed oil, macadamia oil, grapeseed oil, rapeseed oil, coconut oil, groundnut oil, palm oil, castor oil, jojoba oil, olive oil or cereal germ oils; fatty acid esters; alcohols; acetylglycerides; octanoates, decanoates or ricinoleates of alcohols or of polyalcohols; fatty acid triglycerides; glycerides;
fluoro and perfluoro oils,
silicone gums;
waxes of animal, plant, mineral or synthetic origin, such as microcrystalline waxes, paraffin wax, petrolatum, petroleum jelly, ozokerite or montan wax; beeswax, lanolin and derivatives thereof; candelilla wax, ouricury wax, carnauba wax, Japan wax, cocoa butter, cork fiber wax or sugarcane wax; hydrogenated oils that are solid at 25° C., ozokerites, fatty esters and glycerides that are solid at 25° C.; polyethylene waxes and the waxes obtained by Fischer-Tropsch synthesis; hydrogenated oils that are solid at 25° C.; lanolins; fatty esters that are solid at 25° C.; silicone waxes; fluoro waxes.

In a known manner, the composition according to the invention may comprise adjuvants that are common in the field under consideration, such as hydrophilic or lipophilic gelling agents, hydrophilic or lipophilic additives, active agents, especially hydrophilic or lipophilic cosmetic or pharmaceutical active agents, preserving agents, antioxidants, solvents, fragrances, fillers, pigments, nacres, UV-screening agents, odor absorbers and dyes. Depending on their nature, these adjuvants may be introduced into the fatty phase, into the aqueous phase and/or into lipid spherules.

The nature and amount of these adjuvants may be chosen by a person skilled in the art on the basis of his general knowledge, so as to obtain the presentation form desired for the composition. In any case, a person skilled in the art will take care to select all the optional additional compounds and/or the amount thereof such that the advantageous properties of the composition according to the invention are not, or are not substantially, adversely affected by the envisioned addition.

The cosmetic or pharmaceutical compositions according to the invention may especially be in the form of a composition for caring for and/or treating ulcerated areas or areas that have undergone cutaneous stress or microstress, especially generated by exposure to UV and/or contact with an irritant product.

Thus, the compositions according to the invention may especially be in the form of:
a care, treatment, cleansing or protective product for facial or bodily skin, including the scalp, such as a facial or body care composition (day care, night care or moisturizing care); a facial anti-wrinkle or anti-aging composition, a facial matting composition; a composition for irritated skin; a makeup-removing composition; a body milk, especially a moisturizing milk and optionally an after-sun milk;
an antisun composition, artificial tanning (self-tanning) composition or an after-sun care composition;
a haircare composition, and especially an antisun cream or gel; a scalp care composition, especially a hair-loss counteractant or a hair restorer;
a makeup product for the skin of the face, the body or the lips, such as a foundation, a tinted cream, a makeup rouge, an eyeshadow, a loose or compact powder, a concealer stick, a cover stick, a lipstick or a lipcare product.

The compositions according to the invention find a preferred application as compositions for facial skincare, of anti-wrinkle or anti-aging type, and as antisun or after-sun compositions.

The composition used according to the invention may also contain other active agents, and especially at least one compound chosen from: moisturizers; depigmenting agents; anti-glycation agents; NO-synthase inhibitors; agents for stimulating the synthesis of dermal or epidermal macromolecules and/or for preventing their degradation; agents for stimulating fibroblast proliferation and/or for stimulating keratinocyte differentiation; muscle relaxants; tensioning agents; antipollution agents and/or free-radical scavengers, sunscreens and mixtures thereof.

The term “moisturizer” means:
a compound that acts on the barrier function, in order to maintain the moisturization of the stratum corneum, or an occlusive compound. Mention may be made of ceramides, sphingoid-based compounds, lecithins, glycosphingolipids, phospholipids, cholesterol and derivatives thereof, phytosterols (stigmasterol, β-sitosterol or campesterol), essential fatty acids, 1,2-diacylglycerol, 4-chromanone, pentacyclic triterpenes such as ursolic acid, liquid petroleum jelly and lanolin;
or a compound that directly increases the water content of the stratum corneum, such as trehalose and derivatives thereof, hyaluronic acid and derivatives thereof, glycerol, pentanediol, sodium pidolate, serine, xylitol, sodium lactate, polyglyceryl acrylate, ectoin and derivatives thereof, chitosan, oligosaccharides and polysaccharides, cyclic carbonates, N-lauroylpyrrolidonecarboxylic acid and N-α-benzoyl-L-arginine;
or a compound that activates the sebaceous glands, such as DHEA, 7-oxide and/or 17-alkyl derivatives thereof, sapogenins and vitamin D and derivatives thereof.

The term “anti-glycation agent” means a compound that can prevent and/or reduce the glycation of skin proteins, in particular of dermal proteins such as collagen.

Examples of anti-glycation agents are plant extracts of the Ericacea family, such as an extract of blueberry (Vaccinium angustifolium); ergothioneine and its derivatives; and hydroxystilbenes and their derivatives, such as resveratrol and 3,3′,5,5′-tetra-hydroxystilbene.

Examples of NO-synthase inhibitors that are suitable for use in the present invention especially comprise a plant extract of the species Vitis vinifera which is sold especially by the company Euromed under the name “Leucocyanidines® de raisins extra”, or by the company Indena under the name Leucoselect®, or finally by the company Hansen under the name “Extrait de marc de raisin”; a plant extract of the species Olea europaea which is preferably obtained from olive tree leaves and is sold especially by the company Vinyals in the form of a dry extract, or by the company Biologia & Technologia under the trade name Eurol BT; and a plant extract of the species Ginkgo biloba which is preferably a dry aqueous extract of this plant sold by the company Beaufour under the trade name “Ginkgo biloba extrait standard”.

Among the active agents for stimulating dermal macromolecules or for preventing their degradation, mention may be made of those that act:
either on collagen synthesis, such as extracts of Centella asiatica; asiaticosides and derivatives; synthetic peptides such as iamin, biopeptide CL or palmitoyloligopeptide sold by the company Sederma; peptides extracted from plants, such as the soybean hydrolyzate sold by the company Coletica under the trade name Phytokine®; and plant hormones such as auxins and lignans;
or on elastin synthesis, such as the extract of Saccharomyces cerivisiae sold by the company LSN under the trade name Cytovitin®; and the extract of the alga Macrocystis pyrifera sold by the company SECMA under the trade name Kelpadelie®;
or on glycosaminoglycan synthesis;
or on fibronectin synthesis;
or on the inhibition of metalloproteases (MMP), such as, more particularly, MMP 1, 2, 3 or 9. Mention may be made of: retinoids and derivatives, oligopeptides and lipopeptides, lipoamino acids, the malt extract sold by the company Coletica under the trade name Collalift®; extracts of blueberry or of rosemary; lycopene; isoflavones, their derivatives or plant extracts containing them, in particular extracts of soybean (sold, for example, by the company Ichimaru Pharcos under the trade name Flavosterone SB®), of red clover, of flax, of kakkon, or of sage;
or on the inhibition of serine proteases such as leukocyte elastase or cathepsin G. Mention may be made of: the peptide extract of Leguminosa seeds (Pisum sativum) sold by the company LSN under the trade name Parelastyl®; and heparinoids and pseudodipeptides such as (2-[acetyl(3-trifluoromethylphenyl)amino]-3-methyl-butyrylamino)acetic acid.

Among the active agents that stimulate epidermal macromolecules, such as fillagrin and keratins, mention may be made especially of the extract of lupin sold by the company Silab under the trade name Structurine®; the extract of beech Fagus sylvatica buds sold by the company Gattefosse under the trade name Gatuline®; and the extract of the zooplankton Salina sold by the company Seporga under the trade name GP4G®.

The agents for stimulating fibroblast proliferation that may be used in the composition according to the invention may be chosen, for example, from plant proteins or polypeptides, extracted especially from soybean (for example an extract of soybean sold by the company LSN under the name Eleseryl SH-VEG 8® or sold by the company Silab under the trade name Raffermine®); and plant hormones such as giberrellins and cytokinins.

The agents for stimulating keratinocyte proliferation that may be used in the composition according to the invention especially comprise retinoids such as retinol and its esters, including retinyl palmitate; phloroglucinol; extracts of walnut cakes sold by the company Gattefosse; and extracts of Solanum tuberosum sold by the company Sederma.

The agents for stimulating keratinocyte differentiation comprise, for example, minerals such as calcium; the extract of lupin sold by the company Silab under the trade name Photopreventine®; sodium β-sitosteryl sulfate sold by the company Seporga under the trade name Phytocohesine®; and the extract of corn sold by the company Solabia under the trade name Phytovityl®; and lignans such as secoisolariciresinol.

A subject of the present invention is also a cosmetic or therapeutic process for treating bodily or facial skin, including the scalp, in which a cosmetic composition comprising an effective amount of at least one C-glycoside derivative in combination with ascorbic acid or a derivative or analog thereof and more particularly as defined above is applied to the skin, left in contact with the skin and then optionally rinsed off.

The cosmetic treatment process of the invention may be performed especially by applying the cosmetic compositions as defined above according to the usual technique for using these compositions. For example: application of creams, gels, sera, lotions, makeup-removing milks or antisun compositions to the skin or to dry hair; application of a scalp lotion to wet hair.

The invention is illustrated in greater detail in the examples that follow, which are presented as nonlimiting illustrations of the invention.

The efficacy of a combination in accordance with the invention is tested by assaying the procollagen 1. To do this, a composition in accordance with the invention is placed in contact with normal human dermal fibroblasts and the whole is left to incubate for 72 hours before assaying.

The other materials and the protocol used are specified hereinbelow.

The C-glycoside derivative used is C-β-D-xylopyranoside-2-hydroxypropane sold under the name Mexoryl® from Chimex. It is in the form of a solution containing 30% by weight of active material in a 60/40 water/1,2-propanediol mixture.

### EXPERIMENTAL PROTOCOL

Normal human dermal fibroblasts (NHDF) (R8PF2), obtained from a mammary plasty
Type: pool PF2, used at the 8th passage
Culture: 37° C., 5% CO₂
Test culture medium: DMEM (Invitrogen 21969035)
L-glutamine 2 mM (Invitrogen 25030024)
Penicillin 50 IU/ml streptomycin 50 μg/ml (Invitrogen 15070063)
10% Foetal calf serum (v/v, Invitrogen 10270098).

The normal human dermal fibroblasts (R8PF2) are inoculated in whole DMEM medium and preincubated for 24 hours at 37° C. and 5% CO₂. At 80% confluence, the culture medium is replaced with DMEM test medium containing 10% FCS with or without (blank) the products or mixtures to be tested or the reference (TGF-β). The cells are then incubated at 37° C. for 72 hours. Each experimental condition was performed in triplicate.

After incubation, the culture media are harvested and the type I procollagen assay is performed on a sample of medium using a specific ELISA assay kit and according to the supplier's instructions (Procollagen Type I C-Peptide EIA Kit, Bio-Whittaker MK101). An MTT viability test is performed on the cell lawns.

The results are given in the table below:

| | | Procollagen | | | %/ | |
|---|---|---|---|---|---|---|
| Treatment | Concentration | 1 (ng/ml) | SD* | N* | blank | P |
| Control | — | 1922 | 364 | 3 | 100 | <0.01 |
| C-Glycoside | 1 mM | 1838 | 258 | 3 | 96 | >0.05 |
| derivative | | | | | | |
| Ascorbic | 0.01 mg/ml | 17052 | 1414 | 3 | 887 | <0.01 |
| acid | | | | | | |
| Mixture | 1 mM + 0.01 | 20159 | 390 | 3 | 1049 | <0.01 |
| | mg/ml | | | | | |

*N: test number SD: standard deviation

Under these experimental conditions, the amount of procollagen 1 synthesized and secreted by the fibroblasts after 72 hours is correctly detectable. Under the experimental conditions of this test (culturing for 72 hours in DMEM medium containing 10% FCS), it is noted that:
the C-glycoside derivative tested at 1 mm does not significantly modify the amount of procollagen 1 synthesized by the fibroblasts;
ascorbic acid tested at 0.01 mg/ml significantly increases the synthesis of procollagen 1 by the fibroblasts.

The C-glycoside derivative/ascorbic acid mixture increases the synthesis of procollagen 1 more efficiently than in the presence of ascorbic acid alone.

1-21. (canceled)

22. A cosmetic method for preventively or curatively treating the signs of aging of bodily or facial skin, whether it is chronobiological or photoinduced aging comprising at least a step of applying to the skin a synergistic combination of at least one C-glycoside derivative with at least ascorbic acid or a derivative or analog thereof.

23. A cosmetic method for preventively or curatively treating wrinkles and/or fine lines, wizened skin, lack of skin elasticity and/or tonicity, thinning of the dermis, degradation of collagen fibers, flaccid skin, thinned skin and internal degradation of the skin following exposure to ultraviolet radiation comprising at least a step of applying to the skin a synergistic combination of at least one C-glycoside derivative with at least ascorbic acid or a derivative or analog thereof.

24. A cosmetic method for inhibiting the activity of elastases and/or for limiting and/or combating the degradation of elastic fibers comprising at least a step of applying to the skin a synergistic combination of at least one C-glycoside derivative with at least ascorbic acid or a derivative or analog thereof.

25. A cosmetic method for preventing and/or treating the signs of cutaneous aging and/or for stimulating the synthesis of collagen comprising at least a step of applying to the skin a synergistic combination of at least one C-glycoside derivative with at least ascorbic acid or a derivative or analog thereof.

26. The method according to claim 22, in which the C-glycoside derivative corresponds to the general formula (I) below: 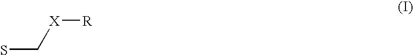
in which:
R represents:
a saturated C₁-C₂₀ or unsaturated C₂-C₂₀ linear alkyl radical, or a saturated or unsaturated, branched or cyclic C₃-C₂₀ alkyl radical;
a saturated C₁-C₂₀ or unsaturated C₂-C₂₀ linear, or saturated or unsaturated, branched or cyclic C₃-C₂₀ hydrofluoroalkyl or perfluoroalkyl radical;
the hydrocarbon-based chain constituting said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
with R₄ and R₅ possibly representing, independently of each other, a hydrogen atom or a saturated C₁-C₃₀ or unsaturated C₂-C₃₀ linear, or a saturated or unsaturated, branched or cyclic C₃-C₃₀ alkyl, perfluoroalkyl or hydrofluoroalkyl radical; or a C₆-C₁₀ aryl radical,
X represents a radical chosen from the groups: 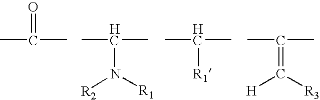
with R₁, R₂ and R₃ representing, independently of each other, a hydrogen atom or a radical R, with R as defined above, and R′₁ represents a hydrogen atom, an —OH group or a radical R as defined above, R₁ possibly also denoting a C₆-C₁₀ aryl radical;
S represents a monosaccharide or a polysaccharide comprising up to 20 sugar units, in pyranose and/or furanose form and of L and/or D series, said mono- or polysaccharide possibly being substituted with a mandatorily free hydroxyl group, and optionally one or more optionally protected amine function(s), and
the bond S—CH₂—X represents a bond of C-anomeric nature, which may be α or β, and also the cosmetically acceptable salts thereof, the solvates thereof and the isomers thereof.

27. The method according to claim 26 in which S represents a monosaccharide chosen from D-glucose, D-xylose, L-fucose, D-galactose and D-maltose.

28. The method according to claim 26 in which X represents a group chosen from —CO—, —CH(OH)— and —CH(NH₂)—.

29. The method according to claim 26 in which R denotes a linear C₁-C₄ radical, optionally substituted with —OH, —COOH or —COOR″₂, R″₂ being a saturated C₁-C₄ alkyl radical.

30. The method according to claim 22 in which the C-glycoside derivative is selected from the group consisting of:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane,
1-(C-β-D-fucofuranosyl)propan-2-one,
1-(C-α-D-fucofuranosyl)propan-2-one,
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one,
C-β-D-maltopyranoside-2-hydroxypropane,
C-α-D-maltopyranoside-2-hydroxypropane,
isomers thereof, and
mixtures thereof.

31. The method according to claim 22 in which the C-glycoside derivative is chosen from C-β-D-xylopyranoside-2-hydroxypropane and C-β-D-xylopyranoside-2-hydroxypropane.

32. The method according to claim 22 in which the ascorbic acid derivatives or analogs are chosen from ascorbic acid saccharide esters, metal salts of phosphoryl ascorbic acid, alkali metal salts thereof, esters thereof and sugars thereof.

33. The method according to claim 22 in which said ascorbic acid or derivative or analog and said C-glycoside derivative are combined in a mole ratio ranging from 1 to 10.

34. A cosmetic and/or dermatological composition comprising, in a physiologically acceptable medium containing an aqueous phase, at least one C-glycoside derivative in combination with at least ascorbic acid or a derivative or analog thereof.

35. An anhydrous cosmetic and/or dermatological composition comprising, in a physiologically acceptable medium, at least one C-glycoside derivative in combination with at least ascorbic acid or a derivative or analog thereof.

36. The composition according to claim 34 comprising from 0.0001% to 25% by weight, of C-glycoside derivative(s) active material relative to the total weight of the composition.

37. The composition according to claim 34 comprising from 0.001% to 20% by weight of ascorbic acid or derivative or analog thereof relative to the total weight of the composition.

38. The composition according to claim 34 which is in the form of:
a product for caring for, treating, cleansing or protecting facial or bodily skin, including the scalp;
a facial anti-wrinkle or anti-aging composition; a facial matting composition; a composition for irritated skin;
a body milk;
an antisun composition, artificial tanning (self-tanning) composition or after-sun care composition;
a haircare composition; a scalp care composition; or
a makeup product for the skin of the face, the body or the lips.

39. The composition according to claim 34 which is in the form of a facial skincare composition, of anti-wrinkle or anti-aging type, or an antisun or after-sun composition.

40. A cosmetic process for treating bodily or facial skin, including the scalp, comprising at least one step that consists in applying to the skin a cosmetic composition as defined in claim 34.
